(19) United States
(12) Patent Application Publication
(10) Pub. No.: US 2022/0325234 A1
(43) Pub. Date: Oct. 13, 2022

(54) BACTERIAL STRAINS, THEIR COMPOSITIONS AND THEIR USE FOR THE TREATMENT OF GASTROINTESTINAL DISORDERS

(71) Applicant: SOFAR S.P.A., Trezzano Rosa (IT)

(72) Inventors: Andrea BIFFI, Trezzano Rosa (MI) (IT); Walter FIORE, Trezzano Rosa (MI) (IT)

(21) Appl. No.: 17/761,843

(22) PCT Filed: Sep. 21, 2020
(86) PCT No.: PCT/IB2020/058774

§ 371 Date: Mar. 18, 2022

(30) Foreign Application Priority Data
Sep. 20, 2019 (IT) 102019000016805

(51) Int. Cl. C12N 1/20; A61K 35/745; A61K 35/747; A61P 1/00

(52) U.S. Cl. CPC C12N 1/205; A61K 35/745; A61K 35/747; A61P 1/00; C12R 2001/245

(57) ABSTRACT

Bacterial strains including B. breve BbIBS01 (DSM 33231), B. breve BbIBS02 (DSM 33232), B. animalis subsp. lactis BIIBS01 (DSM 33233) and L. plantarum LpIBS01 (DSM 33234) are described. Furthermore, compositions are further described comprising a mixture of at least one or more of the aforementioned bacterial strains and, optionally, at least one further bacterial strain, selected from a bacterial strain L. casei DG® (CNCM I-1572) and bacterial strain L. paracasei LPC-S01™ (DSM 26760) and related method for the treatment of gastrointestinal diseases, disorders or symptoms, in particular functional gastrointestinal disorders, such as, irritable bowel syndrome (IBS).

The present invention relates to novel bacterial strains, such as: a strain belonging to the species Bifidobacterium breve identified as Bifidobacterium breve BbIBS01 (DSM 33231), a strain belonging to the species Bifidobacterium breve identified as Bifidobacterium breve BbIBS02 (DSM 33232), a strain belonging to the species Bifidobacterium animalis identified as Bifidobacterium animalis subsp. lactis BIIBS01 (DSM 33233) and a strain belonging to the species Lactobacillus plantarum identified as Lactobacillus plantarum LpIBS01 (DSM 33234).

Furthermore, the present invention relates to compositions comprising a mixture comprising or, alternatively, consisting of: at least one or more bacterial strains selected from the group comprising or, alternatively, consisting of said Bifidobacterium breve BbIBS01 (DSM 33231), Bifidobacterium breve BbIBS02 (DSM 33232), Bifidobacterium animalis subsp. lactis BIIBS01 (DSM 33233) and Lactobacillus plantarum LpIBS01 (DSM 33234), and, optionally, at least one further bacterial strain selected from the group comprising or, alternatively, consisting of: Lactobacillus casei DG® (CNCM I-1572) and Lactobacillus paracasei LPC-S01™ (DSM 26760). Lastly, the present invention relates to said bacterial strains or mixtures thereof or compositions thereof for use in a method for the preventive and/or curative treatment of gastrointestinal diseases, disorders or symptoms in particular functional gastrointestinal disorders such as, for example, irritable Bowel Syndrome (IBS), and/or inflammatory disorders such as, for example chronic inflammatory bowel diseases (IBD).

Irritable bowel syndrome (in short, IBS) belongs to the group of functional gastrointestinal disorders (FGIDs), a diagnostic category that can be defined based on the symptomatic presentation alone and characterised by the absence of an evident pathogenetic substrate. Gastrointestinal functional disorders (FGIDs), also called gut-brain axis disorders, are a group of disorders classified based on gastrointestinal symptoms related to any combination of: bowel motility disorders, visceral hypersensitivity, altered mucosal and immune function, alteration of the intestinal microbiota, alteration of the perception of stimuli in the central nervous system. IBS is one of the most common gastrointestinal disorders, affecting about 15-20% of the population, where abdominal discomfort or pain are associated with changes in the intestinal habitat. Although reported in literature, the evident alterations of the lumen or of the gastrointestinal mucosa at the tissue, cell or molecular level are variable events and they have not been irrefutably identified in the IBS. Altered immune responses appear to be involved, but they cannot explain the symptoms entirely. Similarly, an alteration of the gut microbiota (i.e. a dysbiosis) contributes to pathophysiology, but no specific pathogen or pathobiont has yet been reliably related to the IBS. The irritable bowel syndrome (IBS) is a disorder of the bowel function characterised by abdominal pain related with changes of the small intestinal tract (either constipation-wise or diarrhoeic-wise) and with signs of altered defecation and tympanites. IBS is not to be confused with spastic colitis, given that spastic colitis is an organic disease that arises from the inflammation that causes spasms and abdominal pain. According to Rome IV criteria, IBS is characterised by recurrent abdominal pain, on average, at least 1 day per week in the last 3 months, associated with two or more of the following criteria: related to defecation, associated with a change in the frequency of faeces, associated with a change in the shape (appearance) of faeces. The criteria must be met for the last 3 months with onset of symptoms at least 6 months prior to diagnosis.

Depending on the characteristics of the faeces, four groups in which patients can be stratified are distinguished: IBS with predominant constipation (small bowel constipation), IBS with predominant diarrhoea (small bowel diarrhoea), IBS with alternating constipation and diarrhoea, unclassified IBS.

Currently, the therapies available for the treatment of the IBS are aimed at the resolution of the pathogenetic events underlying the IBS. In subjects with small bowel diarrhoea, the frequency of bowel movements can be decreased by reducing food intake of short chain carbohydrates which are poorly absorbed in the small intestine (FODMAP: Fermentable Oligosaccharides, Disaccharides, Monosaccharides and Polyols) such as fructose, sorbitol and mannitol. It may be useful to combine kaolin-based preparations such as diosmectite with these solutions. Available in subjects with predominantly small bowel constipation and with presence of tympanites, are preparations with low concentrations of polyethylene glycols/mineral salts, to be taken daily. Furthermore, the use of linaclotide, a guanylate cyclase C receptor agonist, is also available in these subjects, who suffer from moderate-severe constipation. The use of anxiolytics (such as benzodiazepines) in the short periods when the patient recognises the anxiety thereof, is useful in reducing psychological participation in pain, with reduction thereof. Similarly, besides directly modulating pain without altering psychic function, the use of antidepressants such as tricyclics and SSRIs (selective serotonin reuptake inhibitors) is capable of improving sleep quality and decreasing the frequency of attacks. Other therapies instead aim at controlling pain; in this sense, some spasmolytics are particularly useful. Anticholinergic-antispasmodic drugs (antimuscarinic drugs), such as for example atropine, scopolamine, mebeverine, are used to reduce gastric secretion and bowel motility. Similarly to the therapy of diverticular disease, tympani syndrome can be reduced by using poorly absorbable antibiotics, such as for example rifaximin, and probiotics that regulate gut flora.

However, the aforementioned treatments often do not allow a complete and lasting resolution of the disease and its symptoms.

The need therefore remains high to provide an effective solution for the treatment of gastrointestinal disorders, in particular functional gastrointestinal disorders, more in particular for irritable bowel syndrome (IBS), small bowel constipation, small bowel diarrhoea, alternating constipation and diarrhoea and unclassified IBS. Furthermore, there is a need to provide an effective solution for the treatment of inflammatory gastrointestinal disorders, such as, for example, chronic inflammatory bowel diseases (IBD).

Following an extensive research and development activity, the Applicant addresses and solves the aforementioned needs by providing: novel strains of isolated bacteria, such as, (I.i) Bifidobacterium breve BbIBS01 (DSM 33231), (I.ii) Bifidobacterium breve BbIBS02 (DSM 33232), (I.iii) Bifidobacterium animalis subsp. lactis BIIBS01 (DSM 33233) and (I.iv) Lactobacillus plantarum LpIBS01 (DSM 33234) (in short, novel bacterial strains (I.i-Iiv) of the invention or (I.i-Iiv)); mixtures (M) comprising at least one or more of said bacterial strains (I.i-Iiv) and, optionally, at least one further bacterial strain selected from the group comprising or, alternatively, consisting of: Lactobacillus casei DG® (CNCM I-1572) and Lactobacillus paracasei LPC-S01™ (DSM 26760) (in short, mixtures (M) of the invention); compositions comprising said mixtures (M) (in short, compositions of the invention); the use of said bacterial strains, mixtures or compositions for the preventive and/or curative treatment of gastrointestinal disorders, preferably functional gastrointestinal disorders, more preferably of the irritable bowel syndrome (IBS) (IBS with diarrhoea, IBS with constipation, IBS with alternating constipation and diarrhoea and unclassified IBS), and/or inflammatory gastrointestinal disorders, more preferably chronic inflammatory bowel diseases (IBD), as reported in the present description and in the claims.

Lactobacillus plantarum LpIBS01 (DSM 33234) was isolated from the human gastrointestinal tract. The Bifidobacteria in question, such as Bifidobacterium breve BbIBS01 (DSM 33231), (I.ii) Bifidobacterium breve BbIBS02 (DSM 33232), (I.iii) Bifidobacterium animalis subsp. lactis BIIBS01 (DSM 33233), are of human origin and they are naturally found in the human intestine.

Specifically, said novel bacterial strains of the invention (I.i-Iiv) or derivatives thereof, mixtures (M) of bacterial strains of the present invention and compositions comprising said mixtures (M) of the present invention, are effective in the preventive and/or curative treatment of gastrointestinal disorders, in particular with regard to functional gastrointestinal disorders, such as for example IBS, given that they determine:
at the level of the gut microbiota, a positive modulation of the microbial populations present, such as for example an increase in the bacterial population of the genus Lactobacillus and of the genus Bifidobacterium and, at the same time, a significant decrease in the bacterial population belonging to the genus Ruminococcus, a pathobiont normally associated with IBS;
an increase in the intestinal concentration of short-chain fatty acids, in particular butyric and/or acetic acid;
a positive modulation of the level of bacterial metabolic products (metabolomics), such as for example free amino acids and biogenic amines;
a positive modulation of the inflammatory pathway, with, for example, a decrease in the pro-inflammatory cytokines, such as IL-6 and/or IL-15 or others;
an improvement in intestinal permeability, evaluated for example through the serum levels of zonulin, citrulline and PV-1;
a positive modulation at the level of the serotoninergic pathway.

Furthermore, the bacterial strains comprised in the mixtures and compositions of the present invention, such as strains belonging to the genus Lactobacillus and Bifidobacterium, produce a variety of beneficial effects given that each species exerts a distinct action on the gastrointestinal tract by means of various mechanisms such as, for example, secreting bacteriocins, modulating the immune system of the host and so on. Such actions may be complementary or synergistic

In addition, said novel bacterial strains (I.i-Iiv), the mixtures (M) of bacterial strains and compositions comprising said mixtures (M) of the present invention affect the expression of different genes involved in the immune responses in the intestine, in particular in the ileum, making their anti-inflammatory/regulatory activity in the intestine plausible. Thus, the bacterial strains, mixtures and compositions of the invention are suitable and effective in the preventive and/or curative treatment of inflammatory gastrointestinal disorders, such as chronic inflammatory bowel diseases (IBD).

Although probiotic bacterial strains have been used in the prior art to improve IBS symptoms, the precise efficacy of probiotics in IBS remains largely unknown. Since probiotic bacterial strains belong to different strains and species, there is no definitive conclusion in the literature on which strain and species are most effective.

Furthermore, the novel bacterial strains, mixtures and compositions of the invention do not have significant adverse effects and they can be administered to all subjects, particularly to paediatric subjects and pregnant women.

Lastly, the mixtures or compositions of the invention are effective, easy to prepare and cost-effective.

These and other objects which will become clear from the detailed description that follows, are achieved by the bacterial strains, by the mixtures and by the compositions of the present invention thanks to the technical characteristics present in the description and claimed in the attached claims.

### DESCRIPTION OF THE FIGURES

FIG. 1: design of the clinical trial with the run-in, treatment, cross-over, treatment and follow-up phases.

### DETAILED DESCRIPTION OF THE INVENTION

Forming an object of the present invention is a bacterial strain belonging to the species Bifidobacterium breve identified as (I.i) Bifidobacterium breve BbIBS01, or a derivative thereof, wherein said bacterial strain was deposited according to the Budapest Treaty, at Deutsche Sammlung von Mikroorganismen und Zellkulturen GmbH (DSMZ) under deposit number DSM 33231 on 31 Jul. 2019 by SOFAR S.p.A. (in short, BbIBS01 or B. breve BbIBS01 DSM 33231 or (I.i)).

Forming an object of the present invention is a bacterial strain belonging to the species Bifidobacterium breve identified as (I.ii) Bifidobacterium breve BbIBS02, or a derivative thereof, wherein said bacterial strain was deposited according to the Budapest Treaty, at Deutsche Sammlung von Mikroorganismen und Zellkulturen GmbH (DSMZ) under deposit number DSM 33232 on 31 Jul. 2019 by SOFAR S.p.A. (in short, BbIBS02 or B. breve BbIBS02 DSM 33232 or (I.ii)).

Forming an object of the present invention is a bacterial strain belonging to the species Bifidobacterium animalis identified as (I.iii) Bifidobacterium animalis subsp. lactis BIIBS01, or a derivative thereof, wherein said bacterial strain was deposited according to the Budapest Treaty, at Deutsche Sammlung von Mikroorganismen und Zellkulturen GmbH (DSMZ) under deposit number DSM 33233 on 31 Jul. 2019 by SOFAR S.p.A. (in short, BIIBS01 or B. animalis subsp. lactis BIIBS01 DSM 33233 or (I.iii)).

Forming an object of the present invention is a bacterial strain belonging to the species Lactobacillus plantarum identified as (I.iv) Lactobacillus plantarum LpIBS01, or a derivative thereof, wherein said bacterial strain was deposited according to the Budapest Treaty, at Deutsche Sammlung von Mikroorganismen and Zellkulturen GmbH (DSMZ) under deposit number DSM 33234 on 31 Jul. 2019 by SOFAR S.p.A. (in short, LpIBS01 or L. plantarum LpIBS01 DSM 33234 or (I.iv)).

Preferably, the bacterial strains of the present invention (i.e. (I.i), (I.ii), (I.iii), (I.iv), (II.i), (II.ii)) are viable bacterial strains (live and viable), such as, for example, viable bacterial strains present in probiotic products or in Live Biotherapeutic Products (in short, LBP, such as pharmaceutical products comprising viable bacterial strains).

“Probiotics” are live and viable micro-organisms (i.e. bacterial strains) which, when administered in adequate amount, confer benefits to the health of the host; the term “probiotics” refers to micro-organisms present in or added to food (FAO and WHO definition).

In the context of the present invention, the term “derivative” or “deirivatives” of a bacterial strain of the present invention (i.e. (I.i), (I.ii), (I.iii), (I.iv), (II.i), (II.ii)) are used to indicate the tyndallized or inactivated bacterial strain, (for example, by means of gamma irradiation or sonication), or lysates or extracts of the bacterial strain (paraprobiotics), or any derivative and/or component of the bacterial strain, preferably exopolysaccharide, parietal fraction, metabolites or metabolic bioproducts generated by the bacterial strain (postbiotics) and/or any other product derived from the bacterial strain. Preferably, the term “derivative” of the bacterial strains of the present invention is used to indicate the tyndalized or inactivated bacteria strain.

According to an aspect of the present invention, in the mixtures (M) of the invention or in the compositions of the invention, part of the bacterial strains can be viable and part of the bacterial strains can be in the form of derivatives, as defined above (e.g. tyndalized).

Forming an object of the present invention is a mixture (M) (in short, mixture (M) of the invention comprising or, alternatively, consisting of at least one or more bacterial strains, or a derivative thereof, selected from the group A comprising or, alternatively, consisting of: (I.i) B. breve BbIBS01 (DSM 33231), (I.ii) B. breve BbIBS02 (DSM 33232), (I.iii) B. animalis subsp. lactis BIIBS01 (DSM 33233), (I.iv) L. plantarum LpIBS01 (DSM 33234), and mixtures thereof.

Said mixture (M) of the invention may comprise a single bacterial strain selected from the group A comprising or, alternatively, consisting of: (I.i), (I.ii), (I.iii) and (I.iv); in short mixture M.1.

Said mixture (M) of the invention may comprise two bacterial strains selected from group M.2 consisting of: (I.i) and (I.ii), (I.i) and (I.iii), (I.i) and (I.iv), (I.ii) and (I.iii), (I.ii) and (I.iv), (I.iii) and (I.iv); in short mixture M.2. Said mixture (M) of the invention may comprise three bacterial strains selected from group M.3 consisting of: (I.i) and (I.ii) and (I.iii), (I.i) and (I.ii) and (I.iv), (I.i) and (I.iii) and (I.iv), (I.ii) and (I.iii) and (I.iv); in short mixture M.3.

Said mixture (M) of the invention may comprise four bacterial strains selected from group M.4 consisting of: (I.i) B. breve BbIBS01 (DSM 33231), (I.ii) B. breve BbIBS02 (DSM 33232), (I.iii) B. animalis subsp. lactis BIIBS01 (DSM 33233) and (I.iv) L. plantarum LpIBS01 (DSM 33234); in short mixture M.4.

In an embodiment of the present invention, besides at least one or more bacterial strains selected from group A, or a derivative thereof, the mixture (M) of the invention comprises at least one further bacterial strain selected from the group B comprising or, alternatively, consisting of: a bacterial straini (II.i) Lactobacillus casei DG® (CNCM I-1572), a bacterial straini (II.ii) Lactobacillus paracasei LPC-S01™ (DSM 26760) and a mixture thereof.

A bacterial strain identified as Lactobacillus casei DG® (trademark registered by SOFAR S.p.A.) was deposited by SOFAR S.p.A. at the National Collection of Cultures of Microorganisms of the Pasteur Institute in Paris under accession number CNCM I-1572 on 5 May 1995 by SOFAR S.p.A. (in short, DG® or L. casei DG® CNCM I-1572 or (II.i)); the strain was initially named Lactobacillus casei DG® sub.casei; it was subsequently reclassified as Lactobacillus paracasei DG®i. It should be observed that it is still and exclusively the same bacterial strain irrespective of the name Lactobacillus casei DG® or Lactobacillus paracasei DG®.

A bacterial strain identified as Lactobacillus paracasei LPC-S01™, alternatively named Lactobacillus paracasei S01, was deposited at Deutsche Sammlung von Mikroorganismen and Zellkulturen GmbH (DSMZ) under accession number DSM 26760 on 15 May 2017 by SOFAR S.p.A. (date of application for conversion of the deposit into a deposit according to the Budapest Treaty; date of original deposit: 11 Jan. 2013) (in short, LPC-S01™ or L. paracasei LPC-S01™ DSM 26760 or (II.ii)). It should be observed that it is still and exclusively the same bacterial strain irrespective of the name Lactobacillus paracasei S01 DSM 26760 or Lactobacillus paracasei LPC-S01™ DSM 26760 used by the Applicant.

In a preferred embodiment of the present invention, besides at least one or more bacterial strains selected from group A, the mixture (M) of the invention further comprises a bacterial strain (II.i) Lactobacillus casei DG® (CNCM I-1572), or derivatives thereof (i.e. Mixture (M)+(II.i), wherein M may be M.1, M.2, M.3 or M.4).

In an embodiment of the present invention, besides at least one or more bacterial strains selected from group A, the mixture (M) of the invention, further comprises a bacterial strain (II.ii) Lactobacillus paracasei LPC-S01™ (DSM 26760), or derivatives thereof (i.e. Mixture (M)+(II.ii), wherein M may be M.1, M.2, M.3 or M.4).

In an embodiment of the present invention, besides at least one or more bacterial strains selected from group A, the mixture (M) of the invention further comprises a bacterial strain (II.i) Lactobacillus casei DG® (CNCM I-1572) and a bacterial strain (II.ii) Lactobacillus paracasei LPC-S01™ (DSM 26760), or derivatives thereof (i.e. mixture (M)+(II.i)(II.ii), wherein M may be M.1, M.2, M.3 or M.4).

According to an aspect of the invention, said mixture (M) of the invention comprises a bacterial strain selected from the group A (or group of mixtures M.1), as defined in the present invention, and a bacterial strain (II.i) Lactobacillus casei DG® (CNCM I-1572), or derivatives thereof (i.e.: (I.i) and (II.i), (I.ii) and (II.i), (I.iii) and (II.i), (I.iv) and (II.i); in short mixture M.1+(II.i).

According to an aspect of the invention, said mixture (M) of the invention comprises two bacterial strains selected from group of mixtures M.2, as defined in the present invention, and a bacterial straini (II.i) Lactobacillus casei DG® (CNCM I-1572), or derivatives thereof (i.e.: (I.i) and (I.ii) and (II.i), (I.i) and (I.iii) and (II.i), (I.i) and (I.iv) and (II.i), (I.ii) and (I.iii) and (II.i), (I.ii) and (I.iv) and (II.i), (I.iii) and (I.iv) and (II.i); in short mixture M.2+(II.i). According to an aspect of the invention, said mixture (M) of the invention comprises three bacterial strains selected from group of mixtures M.3, as defined in the present invention, and a bacterial strain (II.i) Lactobacillus casei DG® (CNCM I-1572), or derivatives thereof (i.e.: (I.i) and (I.ii) and (I.iii) and (II.i), (I.i) and (I.ii) and (I.iv) and (II.i), (I.i) and (I.iii) and (I.iv) and (II.i), (I.ii) and (I.iii) and (I.iv) and (Ii.i); in short mixture M.3+(II.i). In a preferred embodiment of the present invention, said mixture (M) of the invention comprises the four bacterial strains (I.i) B. breve BbIBS01 (DSM 33231), (I.ii) B. breve BbIBS02 (DSM 33232), (I.iii) B. animalis subsp. lactis BIIBS01 (DSM 33233) and (I.iv) L. plantarum LpIBS01 (DSM 33234) and a bacterial strain (II.i) Lactobacillus casei DG® (CNCM I-1572), or derivatives thereof (in short mixture M.4+(II.i).

In an embodiment of the present invention, said mixture (M) of the invention comprises the four bacterial strains (I.i) B. breve BbIBS01 (DSM 33231), (I.ii) B. breve BbIBS02 (DSM 33232), (I.iii) B. animalis subsp. lactis BIIBS01 (DSM 33233) and (I.iv) L. plantarum LpIBS01 (DSM 33234) and a bacterial strain (II.ii) Lactobacillus paracasei LPC-S01™ (DSM 26760), or derivatives thereof (in short mixture M.4+(II.ii).

In an embodiment of the present invention, said mixture (M) of the invention comprises the four bacterial strains (I.i) B. breve BbIBS01 (DSM 33231), (I.ii) B. breve BbIBS02 (DSM 33232), (I.iii) B. animalis subsp. lactis BIIBS01 (DSM 33233) and (I.iv) L. plantarum LpIBS01 (DSM 33234) and a bacterial strain (II.i) Lactobacillus casei DG® (CNCM I-1572) and a bacterial strain (II.ii) Lactobacillus paracasei LPC-S01™ (DSM 26760), or derivatives thereof (in short mixture M.4+(II.i)+(II.ii)).

In the context of the present invention, the term “mixture/s (M) of the invention” are used to indicate the mixtures M.1, M.2, M.3, M.4, M.1+(II.i), M.1+(II.ii), M.1+(II.i)+(II.ii), M.2+(II.i), M.2+(II.ii), M.2+(II.i)+(II.ii), M.3+(II.i), M.3+(II.ii), M.3+(II.i)+(II.ii), M.4+(II.i), M.4+(II.ii), and M.4+(II.i)+(II.ii), as defined in the context of the present invention.

Forming an object of the present invention is a composition (in short, composition of the invention) comprising a mixture (M) comprising or, alternatively, consisting of at least one or more bacterial strains (i.e. two, three or four strains), or a derivative thereof, selected from the group A comprising or, alternatively, consisting of: (I.i) B. breve BbIBS01 (DSM 33231), (I.ii) B. breve BbIBS02 (DSM 33232), (I.iii) B. animalis subsp. lactis BIIBS01 (DSM 33233) and (I.iv) L. plantarum LpIBS01 (DSM 33234), and, optionally, said composition comprises at least one food or pharmaceutical grade additive and/or excipient.

In the context of the present invention, the term “composition/s of the invention” are used to indicate the compositions comprising the mixtures M.1, M.2, M.3, M.4, M.1+(II.i), M.1+(II.ii), M.1+(II.i)+(II.ii), M.2+(II.i), M.2+(II.ii), M.2+(II.i)+(II.ii), M.3+(II.i), M.3+(II.ii), M.3+(II.i)+(II.ii), M.4+(II.i), M.4+(II.ii) and M.4+(II.i)+(II.ii), as defined in the context of the present invention.

In an embodiment, the composition of the invention comprises a mixture (M.1) comprising or, alternatively, consisting of a bacterial strain selected from the group A (or group of mixtures M.1) comprising or, alternatively, consisting of: (I.i), (I.ii), (I.iii) and (I.iv).

In an embodiment, the composition of the invention comprises a mixture (M.2) comprising or, alternatively, consisting of two bacterial strains selected from group of mixtures M.2 as defined in the present invention.

In an embodiment, the composition of the invention comprises a mixture (M.3) comprising or, alternatively, consisting of three bacterial strains selected from group of mixtures M.3 as defined in the present invention. In a preferred embodiment, the composition of the invention comprises a mixture (M.4) comprising or, alternatively, consisting of four bacterial strains, such as: (I.i) B. breve BbIBS01 (DSM 33231), (I.ii) B. breve BbIBS02 (DSM 33232), (I.iii) B. animalis subsp. lactis BIIBS01 (DSM 33233) and (I.iv) L. plantarum LpIBS01 (DSM 33234) (in short, (I.i), (I.ii), (I.iii) and (I.iv)).

In an embodiment of the present invention, the composition of the invention comprises the mixture (M) of the invention which, besides at least one or more bacterial strains selected from group A, comprises at least one further bacterial strain selected from the group B comprising or, alternatively, consisting of: a bacterial strain (II.i) Lactobacillus casei DG® (CNCM I-1572) and a bacterial strain (II.ii) Lactobacillus paracasei LPC-S01™ (DSM 26760), such as mixture M+(II.i) or mixture M+(II.ii) or mixture M+(II.i)+(II.ii), wherein M may be M.1, M.2, M.3 or M.4 as defined in the context of the present invention.

In a preferred embodiment of the present invention, the composition of the invention comprises the mixture (M) of the invention which, besides at least one or more bacterial strains selected from group A, further comprises a bacterial strain (II.i) Lactobacillus casei DG® (CNCM I-1572), or derivatives thereof (i.e. mixture (M)+(II.i), wherein M may be M.1, M.2, M.3 or M.4).

In an embodiment of the present invention, the composition of the invention comprises the mixture (M) of the invention which, besides at least one or more bacterial strains selected from group A, further comprises a bacterial strain (II.ii) Lactobacillus paracasei LPC-S0™ (DSM 26760), or derivatives thereof (i.e. mixture (M)+(II.ii), wherein M may be M.1, M.2, M.3 or M.4).

In an embodiment of the present invention, the composition of the invention comprises the mixture (M) of the invention which, besides at least one or more bacterial strains selected from group A, further comprises a bacterial strain (II.i) Lactobacillus casei DG® (CNCM I-1572) and a bacterial strain (II.ii) Lactobacillus paracasei LPC-S01™ (DSM 26760), or derivatives thereof (i.e. mixture (M)+(II.i)(II.ii), wherein M may be M.1, M.2, M.3 or M.4).

According to an aspect of the invention, the composition of the invention comprises said mixture (M.1+(II.i) of the invention comprising a bacterial strain selected from the group A (or group of mixtures M.1), as defined in the present invention, and a bacterial strain (II.i) Lactobacillus casei DG® (CNCM I-1572).

According to an aspect of the invention, the composition of the invention comprises said mixture (M.2+(II.i) of the invention comprising two bacterial strains selected from group of mixtures M.2, as defined in the present invention and a bacterial strain (II.i) Lactobacillus casei DG® (CNCM I-1572).

According to an aspect of the invention, the composition of the invention comprises said mixture (M.3+(II.i) of the invention comprising three bacterial strains selected from group of mixtures M.3, as defined in the present invention, and a bacterial strain (II.i) Lactobacillus casei DG® (CNCM I-1572).

In a preferred embodiment of the present invention, the composition of the invention comprises said mixture ((M.4)+(II.i) of the invention comprising the four bacterial strains (I.i) B. breve BbIBS01 (DSM 33231), (I.ii) B. breve BbIBS02 (DSM 33232), (I.iii) B. animalis subsp. lactis BIIBS01 (DSM 33233) and (I.iv) L. plantarum LpIBS01 (DSM 33234) and a bacterial strain (II.i) Lactobacillus casei DG® (CNCM I-1572).

In an embodiment of the present invention, the composition of the invention comprises said mixture ((M.4)+(II.i) of the invention comprising or, alternatively, consisting of: the bacterial strains (I.i) B. breve BbIBS01 (DSM 33231), (I.ii) B. breve BbIBS02 (DSM 33232), (I.iii) B. animalis subsp. lactis BIIBS01 (DSM 33233) and (I.iv) L. plantarum LpIBS01 (DSM 33234) and a bacterial strain (11.0 Lactobacillus paracasei LPC-S01™ (DSM 26760). According to an aspect of the invention, the composition of the invention comprises said mixture ((M.4)+(II.i)+(II.i) of the invention comprising the four bacterial strains (I.i), (I.ii), (I.iii) and (I.iv) and the bacterial strains (II.i) Lactobacillus casei DG® (CNCM I-1572) and (II.ii) Lactobacillus paracasei LPC-S01™ (DSM 26760).

Preferably, said bacterial strains (i.e. (I.i), (I.ii), (I.iii), (I.iv), (II.i) and/or (II.ii)) are comprised, independently of each other, in the mixtures (M) of the invention at a concentration comprised in the range from 1×10⁶ CFU to 1×10¹² CFU, preferably from 1×10⁷ CFU to 1×10¹¹ CFU, more preferably from 1×10⁸ CFU to 1×10¹⁰ CFU, for example 1×10⁹ CFU, with respect to the daily intake (CFU: Colony Forming Unit).

In a preferred embodiment, the mixture M.4 or M.4+(II.i) or M.4+(II.ii) or M.4+(II.i)+(II.ii) or the relative compositions of the invention comprise, independently of each other, each of the bacterial strains (I.i), (I.ii), (I.iii), (I.iv) and, optionally, (II.i) and/or (II.i) at a concentration comprised in the range from 1×10⁶ CFU to 1×10¹² CFU, preferably from 1×10⁷ CFU to 1×10¹¹ CFU, more preferably from 1×10⁸ CFU to 1×10¹⁹ CFU, for example 1×10⁹ CFU, with respect to the daily intake.

Preferably, in the mixture M.2 or M.1+(11.0 or M.1+(II.ii) the bacterial strains are at a ratio of about 1:1, in the mixture M.3 or M.2+(II.i) or M.2+(II.ii) the bacterial strains are at a ratio of about 1:1:1, in the mixture M.4 or M.3+(II.i) or M.3+(II.ii) the bacterial strains are at a ratio of about 1:1:1:1, in the mixture M.4+(II.i) or M.4+(II.ii) the bacterial strains are at a ratio of about 1:1:1:1:1, in the mixture M.4+(II.i)+(II.ii) the bacterial strains are at a ratio of about 1:1:1:1:1:1; wherein said ratios are with respect to the CFU.

In an embodiment, besides one of said mixtures (M) of the invention (preferably M4 or M.4+(II.i) or M.4+(II.i), the composition of the present invention may comprise at least one further active component selected from the group comprising or, alternatively, consisting of: other viable and/or parabiotic and/or postbiotic and/or lysed and/or tyndalized and/or inactivated bacterial strains, enzymes, substances having direct or indirect antacid action, prebiotic substances, probiotic substances belonging to the families of yeasts and bacteria, immunostimulatory substances, anti-diarrhoea substances, nutrients, vitamins of group B, C, D, E, organic and/or inorganic salts of magnesium, selenium, zinc, melatonin, valerian, passion flower, lemon balm, hawthorn, chamomile, hop plant, antioxidants, anti-radical agents.

The composition of the invention may be in solid form, such as tablet, chewable tablet, tablet to be dissolved in the mouth or mouth-soluble, capsule, lozenge, granules, flakes or powder (granules or powder to be dissolved in a liquid or mouth-soluble granules or powder, for example mouth-soluble stick), in semi-solid form, such as soft-gel, cream, or in liquid form, such as solution, suspension, dispersion, emulsion or syrup.

The composition of the invention may be formulated for oral (or gastroenteric), sublingual (or buccal), transmucosal, transdermal and/or topical use (or administration), such as rectal, cutaneous, vaginal; it is advantageously formulated for oral use.

The composition of the invention, comprising or, alternatively, consisting of one of said mixtures (M) of the invention (preferably M.4 or M.4+(II.i) or M.4+(II.i), further, optionally, comprises said at least one pharmaceutical or food grade additive and/or excipient, i.e. a substance devoid of therapeutic activity suitable for pharmaceutical or food use. In the context of the present invention additives and/or excipients acceptable for pharmaceutical or food use comprise all auxiliary substances known to the man skilled in the art for the preparation of compositions in solid, semi-solid or liquid form, such as, for example, diluents, solvents (including water, glycerine, ethyl alcohol), solubilisers, acidifiers, thickeners, sweeteners, flavour enhancers, colouring agents, lubricants, surfactants, preservatives, pH stabilising buffers and mixtures thereof.

The composition of the invention, comprising or, alternatively, consisting of one of said mixtures (M) of the invention (preferably M.4 or M.4+(II.i) or M.4+(II.ii), may be a pharmaceutical composition (or Live Biotherapeutic Products), a medical device composition, a dietary supplement, a food or novel food or probiotic product, a cosmetic composition, a composition for a food for special medical purposes (FSMP).

In the context of the present invention, the expression “medical device” is used in the meaning according to the Italian Legislative Decree no 46 dated 24 Feb. 1997 or according to the new Medical Device Regulation (EU) 2017/745 (MDR).

Forming a further object of the present invention are said mixtures (M) of the invention (preferably M.4 or M.4+(II.i) or M.4+(II.ii)) or said compositions of the invention comprising one of said mixtures (M) of the invention (preferably M.4 or M.4+(II.i) or M.4+(II.ii)) for use as medicament.

Said mixtures (M) or said compositions of the invention may also be for use as medicament as adjuvants of further therapeutic approaches, preferably of the pharmacological or dietary type.

In an embodiment, the bacterial strains of the invention, the mixtures (M) of the invention (preferably M.4 or M.4+(II.i) or M.4+(II.i) or the compositions of the invention are for use in a method for the preventive and/or curative treatment of gastrointestinal diseases, disorders or symptoms in a subject in need, preferably of functional gastrointestinal disorders, such as the irritable bowel syndrome (IBS), dyspepsia, pyrosis, oesophagus, stomach and duodenum disorders, SIBO (small intestinal bacterial overgrowth), disorders with sub-inflammatory conditions, preferably wherein said sub-inflammatory conditions are in an elderly subject or in a subject with diverticular disease.

In a preferred embodiment, the bacterial strains of the invention, the mixtures (M) of the invention (preferably M.4 or M.4+(II.i) or M.4+(II.i) or the compositions of the invention are for use in a method for the preventive and/or curative treatment of disorders or symptoms of the irritable bowel syndrome (IBS), constipation, diarrhoea, alternating constipation and diarrhoea and unclassified IBS. Specific examples of disorders or symptoms of the irritable bowel syndrome (IBS) that can be treated by the bacterial strains of the invention, the mixtures (M) of the invention or the compositions of the invention are: intermittent abdominal pain, in the form of cramps, with varying intensity and localisation; flatulence; tympanites; bloating sensation.

In a preferred embodiment of the invention, the mixture M.4+(II.i) or the composition comprising the mixture M.4+(II.i), wherein said mixture M.4+(II.i) comprises or, alternatively consists of bacterial strains (I.i) B. breve BbIBS01 (DSM 33231), (I.ii) B. breve BbIBS02 (DSM 33232), (I.iii) B. animalis subsp. lactis BIIBS01 (DSM 33233) e (I.iv) L. plantarum LpIBS01 (DSM 33234) and (II.i) Lactobacillus casei DG® (CNCM I-1572), is for use in a method for the preventive and/or curative treatment of disorders or symptoms of the irritable bowel syndrome (IBS), constipation, diarrhoea, alternating constipation and diarrhoea and unclassified IBS.

In an alternative embodiment of the invention, the mixture M.4+(II.ii) or the composition comprising the mixture M.4+(II.ii), wherein said mixture M.4+(II.ii) comprises or, alternatively consists of bacterial strains (I.i) B. breve BbIBS01 (DSM 33231), (I.ii) B. breve BbIBS02 (DSM 33232), (I.iii) B. animalis subsp. lactis BIIBS01 (DSM 33233) and (I.iv) L. plantarum LpIBS01 (DSM 33234) and (II.ii) Lactobacillus paracasei LPC-S01™ (DSM 26760), is for use in a method for the preventive and/or curative treatment of disorders or symptoms of the irritable bowel syndrome (IBS), constipation, diarrhoea, alternating constipation and diarrhoea and unclassified IBS.

In an embodiment, the bacterial strains of the invention, said mixtures (M) of the invention (preferably M.4 or M.4+(II.i) or M.4+(II.i) or said compositions of the invention are for use in the treatment of inflammatory gastrointestinal diseases, disorders or symptoms in a subject in need, such Helicobacter pylori, peptic or gastric ulcer, duodenal ulcer, chronic inflammatory bowel diseases (IBD), such as Crohn's disease and ulcerative colitis, microscopic colitis, diverticular disease and diverticulitis; preferably chronic inflammatory bowel diseases (IBD), Crohn's disease, ulcerative colitis, microscopic colitis, diverticular disease, or diverticulitis.

In an embodiment, the bacterial strains of the invention, said mixtures (M) of the invention (preferably M.4 or M.4+(II.i) or M.4+(II.ii)) or said compositions of the invention are for use as immunomodulatory agents (capable of modulating the immune system) and/or immunostimulatory agents for the subject to whom they are administered. Thus, the bacterial strain, or a derivative thereof, and the compositions of the present invention have a valid application for the preventive or curative treatment, of diseases related with alterations of the immune system, in particular, autoimmune diseases and allergies, immunodeficiency diseases, diseases affecting the skin, such as acne, atopic dermatitis.

Advantageously, the bacterial strains of the invention, the mixtures (M) and the compositions of the present invention are capable of positively modulating the inflammatory pathway and, thus, the ratio between inflammatory cytokines and anti-inflammatory cytokines. In particular, the bacterial strains of the invention, the mixtures (M) of the present invention and the compositions of the present invention are capable of reducing the production of pro-inflammatory cytokines, preferably IL-6, IL-15, IL-12 and TNF-α, and/or increasing the production of anti-inflammatory cytokines, preferably IL-10.

Thus, in an embodiment of the present invention, the bacterial strains of the invention, the mixtures (M) of the invention (preferably M.4 or M.4+(II.i) or M.4+(II.ii)) and the compositions of the invention are for use in a method for the preventive and/or curative treatment of diseases or symptoms or disorders caused by or related with/accompanied by an increase in pro-inflammatory cytokines and/or a decrease in anti-inflammatory cytokines, preferably diseases affecting: locomotor system (muscular and skeletal system), digestive system, urogenital system (urinary system and genital system), respiratory system, integumentary system, immune system and/or circulatory system.

In an embodiment, the bacterial strains of the invention, said mixtures (M) of the invention (preferably M.4 or M.4+(II.i) or M.4+(II.ii)) or said compositions of the invention are for use in a method for the preventive or curative treatment of inflammatory musculoskeletal, rheumatological, inflammatory articular diseases and inflammatory post-surgery diseases, preferably for use in methods for the treatment of osteoarthritis, rheumatoid arthritis and ankylosing spondylitis, in particular osteoarthritis of the knee and osteoarthritis of the joints in general.

Forming an object of the present invention is a method for the preventive or curative treatment of gastrointestinal diseases, disorders or symptoms, in particular functional or inflammatory gastrointestinal disorders, preferably IBS (constipation, diarrhoea, alternating constipation and diarrhoea and unclassified IBS) or IBD, which provides for the administration of a therapeutically effective amount of one of the mixtures (M) of the invention (preferably M.4 or M.4+(II.ii)) or M.4+(II.ii)) or of the compositions of the invention to a subject in need.

For the sake clarity, in order to achieve the object of the present invention, the components (or active components) of the mixture (M) of the invention, such as the bacterial strains in the present invention, may be administered separately (preferably within a time interval ranging from 30 minutes to 60 minutes) and in any order but, preferably, the bacterial strains are administered to a subject simultaneously, even more preferably in a single composition to obtain a more rapid effect and ease of administration. When the components (or active components) of the mixture (M) of the invention, such as the bacterial strains, are administered in a single composition, said single composition corresponds to the composition of the present invention.

In the context of the present invention, the expression “subjects” is used to indicate human subjects or animal subjects (e.g. pets, such as dogs or cats or other mammals). Preferably, the compositions of the invention are for use in treatment methods for human subjects.

Unless otherwise specified, the expression composition or mixture or other comprising a component at an amount “comprised in a range from x to y” is used to indicate that said component may be present in the composition or mixture or extract or other at all amounts present in said range, even if not specified, extremes of the range comprised.

The expression “therapeutically effective amount” refers to the amount of composition, mixture and/or bacterial strain that elicits the biological or medicinal response in a tissue, system, mammal, or human being that is sought and defined by an individual, researcher, veterinarian, physician, or other clinician or health worker. In the context of the present invention the term “novel food” is used in its meaning according to the EU Regulation 2015/2283 of 25.11.2015.

Embodiments (FRn) of the present invention are reported hereinafter.

FR1. A composition comprising a mixture M comprising, or alternatively, consisting of at least one bacterial strain selected from the group A comprising, or alternatively, consisting of:
a bacterial strain belonging to the species Bifidobacterium breve identified as Bifidobacterium breve BbIBS01 and having deposit number DSM 33231,
a bacterial strain belonging to the species Bifidobacterium breve identified as Bifidobacterium breve BbIBS02 and having deposit number DSM 33232,
a bacterial strain belonging to the species Bifidobacterium animalis identified as Bifidobacterium animalis subsp. lactis BIIBS01 and having deposit number DSM 33233,
a bacterial strain belonging to the species Lactobacillus plantarum identified as Lactobacillus plantarum LpIBS01 and having deposit number DSM 33234, and mixtures thereof,
   wherein all said bacterial strains were deposited at Deutsche Sammlung von Mikroorganismen and Zellkulturen GmbH (DSMZ) on 31 Jul. 2019 by SOFAR S.p.A.;
   and wherein, optionally, said composition comprises at least one food or pharmaceutical grade additive and/or excipient.

FR2. A composition according to FR1, wherein said mixture M comprises or, alternatively consists of bacterial strains:
Bifidobacterium breve BbIBS01 DSM 33231,
Bifidobacterium breve BbIBS02 DSM 33232,
Bifidobacterium animalis subsp. lactis BIIBS01 DSM 33233, and
Lactobacillus plantarum LpIBS01 DSM 33234

FR3. A composition according FR 1 or 2, wherein said mixture M further comprises at least one further bacterial strain selected from the group B comprising or, alternatively, consisting of: a bacterial strain identified as Lactobacillus casei DG® deposited at the National Collection of Cultures of Microorganisms Pasteur Institute in Paris under accession number CNCM I-1572 on 5 May 1995 by SOFAR S.p.A., a bacterial strain identified as Lactobacillus paracasei LPC-S01™ deposited at Deutsche Sammlung von Mikroorganismen and Zellkulturen GmbH (DSMZ) under accession number DSM 26760 on 15 May 2017 by SOFAR S.p.A. and a mixture thereof.

FR4. A composition according to FR3, wherein said mixture M comprises or, alternatively consists of bacterial strains:
Bifidobacterium breve BbIBS01 DSM 33231,
Bifidobacterium breve BbIBS02 DSM 33232,
Bifidobacterium animalis subsp. lactis BIIBS01 DSM 33233,
Lactobacillus plantarum LpIBS01 DSM 33234, and
Lactobacillus casei DG® CNCM I-1572.

FR5. A composition according to FR3, wherein said mixture M comprises or, alternatively consists of bacterial strains:
Bifidobacterium breve BbIBS01 DSM 33231,
Bifidobacterium breve BbIBS02 DSM 33232,
Bifidobacterium animalis subsp. lactis BIIBS01 DSM 33233,
Lactobacillus plantarum LpIBS01 DSM 33234, and
Lactobacillus paracasei LPC-S01™ DSM 26760.

FR6. The composition according to any one of FR 1 to 5, wherein said composition is formulated for oral, sublingual or buccal, transmucosal, topical, rectal, topical cutaneous or topical vaginal use; preferably said composition is formulated for oral use.

FR7. The composition according to any one of FRs 1 to 6 for use as medicament.

FR8. The composition according to any one FRs 1 a 6 for use in a method for the preventive and/or curative treatment of functional gastrointestinal diseases, disorders and/or symptoms.

FR9. The composition according to claim FR8, wherein said composition is for use in a method for the preventive and/or curative treatment of functional gastrointestinal diseases, disorders and/or symptoms selected from the group comprising or, alternatively, consisting of: irritable bowel syndrome (IBS), dyspepsia, pyrosis, oesophageal disorders, stomach disorders and duodenum disorders, small intestinal bacterial overgrowth (SIBO), gastrointestinal disorders with sub-inflammatory conditions; preferably the irritable bowel syndrome (IBS) with constipation or irritable bowel syndrome (IBS) with diarrhoea or irritable bowel syndrome (IBS) with alternating constipation and diarrhoea.

FR10. The composition according to any one of FRs 1 to 6 for use in a method for the preventive and/or curative treatment of inflammatory gastrointestinal diseases, disorders and/or symptoms.

FR11. The composition according to FR10, wherein said composition is for use in a method for the preventive or curative treatment of inflammatory gastrointestinal disorders selected from the group comprising or, alternatively, consisting of: chronic inflammatory bowel diseases (IBD), Crohn's disease, ulcerative colitis, microscopic colitis, diverticular disease, diverticulitis, Helicobacter pylori, peptic or gastric ulcer, and duodenal ulcers; preferably selected from chronic inflammatory bowel diseases (IBD), Crohn's disease, ulcerative colitis, microscopic colitis, diverticular disease, and diverticulitis.

FR12. A bacterial strain belonging to the species Bifidobacterium breve identified as Bifidobacterium breve BbIBS01, wherein said bacterial strain was deposited, according to the Budapest Treaty, at Deutsche Sammlung von Mikroorganismen und Zellkulturen GmbH (DSMZ) under deposit number DSM 33231 on 31 Jul. 2019 by SOFAR S.p.A.

FR13. A bacterial strain belonging to the species Bifidobacterium breve identified as Bifidobacterium breve BbIBS02, wherein said bacterial strain was deposited at Deutsche Sammlung von Mikroorganismen und Zellkulturen GmbH (DSMZ) under deposit number DSM 33232 on 31 Jul. 2019 by SOFAR S.p.A.

FR14. A bacterial strain belonging to the species Bifidobacterium animalis identified as Bifidobacterium animalis subsp. lactis BIIBS01, wherein said bacterial strain was deposited at Deutsche Sammlung von Mikroorganismen und Zellkulturen GmbH (DSMZ) under deposit number DSM 33233 on 31 Jul. 2019 by SOFAR S.p.A.

FR15. A bacterial strain belonging to the species Lactobacillus plantarum identified as Lactobacillus plantarum LpIBS01, wherein said bacterial strain was deposited at Deutsche Sammlung von Mikroorganismen und Zellkulturen GmbH (DSMZ) under deposit number DSM 33234 on 31 Jul. 2019 by SOFAR S.p.A.

### Experimental Part

Evaluation of the efficacy of the combination of 5 viable bacterial strains in the treatment of patients suffering from irritable bowel syndrome (IBS); multicentre, randomised, double-blind, parallel group, placebo-controlled trial.

### 1. Objectives of the Trial

Primary objective to evaluate the effect of a combined supplement of 5 probiotic strains according to the invention (Composition 1) on the gut microbiota of patients suffering from IBS.

Secondary Objectives

1. To evaluate the effect of Composition 1 on the symptoms of IBS;
2. To evaluate the effect of Composition 1 on abdominal pain/discomfort;
3. To evaluate the effect of Composition 1 on the daily bowel habits of the patient suffering from IBS;
4. To evaluate the effect of Composition 1 on the intestinal permeability
5. To evaluate the recovery of strains of Composition 1 in stool samples;
6. To evaluate the overall satisfaction of the treatment;
7. To evaluate the change in the quality of life of the patients;
8. To evaluate the intake of rescue medication.

In other words, the trial aims at evaluating:
effect of the combination of 5 viable bacterial strains (Composition 1, according to the present invention) on abdominal symptoms in non-constipated patients suffering from irritable bowel syndrome (IBS);
relief of IBS symptoms;
daily consistency of the faeces;
overall satisfaction with the treatment;
quality of life;
psychological impairment;
intake of rescue medication;
composition of the gut microbiota and metabolic products (metabolomics)—and, therefore, modifications at the level of the gut microbial ecosystem;
intestinal permeability;
recovery of strains in faeces;
modifications of the inflammatory pathway.

The possibility of analysing microbiota in blood will be considered as an exploratory objective for the purposes of the trial.

### 2. Experimental Design

Multicentre, randomised, double-blind, parallel group, placebo-controlled trial.

The estimated recruitment period will be approximately 12 months.

Patients will be recruited after signing informed consent prior to any other trial procedure and after evaluating the inclusion/exclusion criteria.

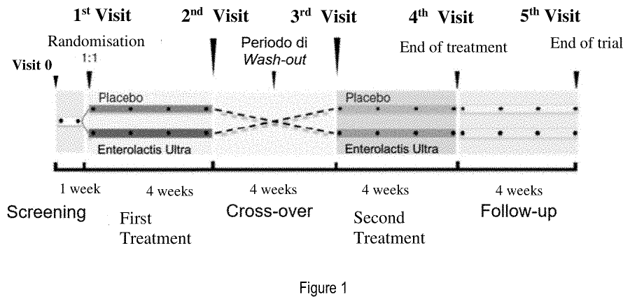

6 trial visits, each of which will be conducted according to the requirements reported in the protocol (see FIG. 1), will be scheduled for each subject.

Trial visits will be conducted as follows: at Visit 0 (screening), at Visit 1 (baseline/beginning of 1st treatment period), at Visit 2 (end of 1st treatment period/start of wash-out), at Visit 3 (end of wash out/beginning of 2nd treatment period), at Visit 4 (end of 2nd treatment period) and at Visit 5 (end of trial).

Subjects will be recruited at the screening visit and they will enter a 1-week screening period.

Thus, if eligible, they will be subjected to a randomising procedure at the baseline visit and they will be randomly assigned to a treatment group (Composition 1/placebo or placebo/Composition 1). Each subject will be provided with the active product (Composition 1) or the placebo for 4 weeks; after a 4-week wash-out period, each subject will enter the second treatment period for another 4 weeks. After visit 4 (end of treatment), patients will enter a four-week follow-up phase until the end of trial visit (visit 5).

Each subject will take 1 sachet of the active product (Composition 1) or Placebo once a day for 4 weeks.

Thus, two treatment sequences can be identified:
Sequence 1 (E-P): patients treated with Composition 1 (E) in the first treatment period and Placebo (P) in the second.
Sequence 2 (P-E): patients treated with Placebo (P) in the first treatment period and Composition 1 (E) in the second.

### 3. Patients

Male or female patients 18 years and 65 years of age, diagnosed with IBS without constipation (see below) according to Rome IV criteria.

The diagnostic criterion for IBS, met for the previous 3 months with onset of symptoms at least 6 months prior to diagnosis, is recurrent abdominal pain at least 1 day per week, associated with 2 or more of the following:
1) Related to defecation;
2) Associated with the change in stool frequency;
3) Associated with the change in the form (appearance) of faeces;
   Patients with IBS without constipation include:
1) IBS with predominant diarrhoea (IBS-D): more than one-quarter (25%) of bowel movements in the form of faeces (Bristol) type 1 or 2.
2) IBS with mixed bowel habits (IBS-M): more than one-quarter (25%) of bowel movements in the form of faeces (Bristol) type 1 or 2 and more than one-quarter (25%) of bowel movements in the form of faeces (Bristol) type 6 or 7.

### 4. Inclusion Criteria

≥18 and ≤65 years of age.
Positive diagnosis of IBS without constipation (IBS-D and IBS-M, both male and female) according to the Rome IV criteria.
Colonoscopy conducted within 5 years prior to the screening visit if the patient is at least 50 years of age negative, or if the patient has one of these alarm signals:
   1) Has a significant recorded weight loss in the last 6 months; or
   2) Shows symptoms at night; or
   3) Has a family history of colon cancer; or
   4) has blood mixed with faeces (excluding blood from haemorrhoids).
Further relevant screening or consultations, where appropriate, negative.
Ability to comply with the trial protocol.

### 5. Exclusion Criteria

Patients suffering from IBS-C or IBS-U according to the Rome IV criteria.
Presence of any relevant organic, systemic or metabolic disease (particularly significant history of cardiac, renal, neurological, psychiatric, oncological, endocrinological, metabolic or hepatic diseases), or abnormal laboratory values detected during the run-in period, considered clinically significant based on predefined values (e.g. renal or hepatic function levels 2 times higher than the highest reference values).
   Established organic bowel diseases, including food allergies or inflammatory bowel diseases (Crohn's disease, ulcerative colitis, diverticular disease, infectious colitis, ischemic colitis, microscopic colitis).
   Previous major abdominal surgery.
Active malignancy of any type, or history of malignancy (patients with a clinical history of other neoplasms that have been surgically removed and that have no evidence of relapse for at least five years prior to trial recruitment are acceptable).
   Untreated food intolerance as established.
   Suspected lactose intolerance, as defined by the anamnestic evaluation or, where appropriate, lactose breath test.
   Use of probiotics or topical/systemic antibiotic therapy over the last month.
   Frequent or systematic use of contact laxatives.
   Pregnant women or women of reproductive age in the absence of effective contraceptive methods.
   Inability to comply with the Protocol.
   Treatment with any experimental drug within the previous 30 days.
   Recent history or suspicion of abuse of alcohol or drug addiction.
   Presence of red or white flags according to the Rome IV criteria.
   Psychosocial Alarm Questionnaire for Functional gastrointestinal Disorders.

### 6. Treatment

Composition 1 according to the present invention comprises: Lactobacillus casei DG®, Lactobacillus plantarum LpIBS01, Bifidobacterium breve BbIBS01, Bifidobacterium breve BbIBS02, Bifidobacterium animalis sub. lactis BIIBS01), 1 dose (capsule or sachet) per day for 4 weeks (approximately 25-30×10⁹ CFU/dose, approximately 5×10⁹ CFU for each bacterial strain).
Composition 2 (placebo), indistinguishable from Composition 1, one capsule/sachet per day for 4 weeks.

### 7. Randomisation

Recruitable patients will enter a 1-week run-in phase and then they will be randomly assigned with a 1:1 ratio to the treatment with Composition 1 (Composition according to the invention) or the bacteria-free equivalent product (Placebo), similar in colour and flavour, once per day for 4 weeks. The clinical trial design with the run-in, treatment, cross-over, treatment and follow-up phases are reported in FIG. 1.

### 8. Endpoints

### Primary Endpoint

Evaluation of changes in faecal microbiota with respect to the baseline in terms of microbial diversity indices (α and β diversity), relative taxonomic abundance and metabolites.

### Secondary End-Points

1. Relief from IBS symptoms, evaluated by means of IBS symptom severity scale (IBS-SSS) score, evaluated at V1, V2, V3, V4 and V5;
2. Evaluation of the daily abdominal pain/discomfort evaluated by means of the standard 11-point numerical rating scale (NRS) (from 0=none to 10=worst possible pain);
3. Evaluation of the frequency and consistency of daily faeces as evaluated by the Bristol Stool Form Scale (BSFS);
4. Change of intestinal permeability (blood sample), with evaluation of zonulin serum, citrulline and PV-1 levels through the ELISA test;
5. Detection of the strains present in Composition 1 in stool samples using qPCR specific primers;
6. Overall satisfaction for the treatment evaluated by means of VAS scale;
7. Evaluation of the quality of life evaluated by means of the Short-Form 12 Items (SF-12) validated health survey on a 0 to 100 scale;
8. Change of the intake of rescue medications.

### Safety Endpoints

9. Collection of adverse events of patients with IBS and serious adverse events during the trial;
10. Concomitant medications during the trial.

### Exploratory Endpoint

Evaluation of changes in blood microbiota with respect to the baseline in terms of microbial diversity indices (α and β diversity), relative taxonomic abundance.

### 9. Population for Analysis and Statistical Considerations

Full Analysis Set (FAS): consists of all randomised patients.
Safety set: consists of all randomised patients who receive at least one trial treatment dose and have at least one post-baseline safety assessment.
Protocol Set (PP): consists of all randomised patients who complete the trial without any significant violation of protocol.
Intention to Treat Set (ITT): consists of all randomised patients who receive at least one trial treatment dose and have at least one post-baseline efficacy assessment.

The primary endpoint of the trial will be descriptively compared between the two trial arms (Composition 1 and Placebo). An appropriate inferential analysis will be carried out solely for exploratory purposes. In general, all variables will be descriptively analysed as concerns the treatment arm and visit (mean, median, standard deviation, minimum and maximum for continuous variables after control of distribution normality by means of Kolmogorov-Smirnov test, frequency distribution for categorical variables). All analyses will be detailed in the Statistical analysis plan (SAP) which will be finalised in version 1.0 prior to the Data Base Lock (DBL). In detail, safety and tolerability data will include (at least) physical examinations, vital signs, laboratory data and adverse events.

1. A composition comprising a mixture M comprising at least one bacterial strain selected from group A comprising:
a bacterial strain belonging to the species Bifidobacterium breve identified as Bifidobacterium breve BbIBS01 and deposited at Deutsche Sammlung von Mikroorganismen und Zellkulturen GmbH (DSMZ) on 31 Jul. 2019 by SOFAR S.p.A.; with deposit number DSM 33231,
a bacterial strain belonging to the species Bifidobacterium breve identified as Bifidobacterium breve BbIBS02 and deposited at Deutsche Sammlung von Mikroorganismen und Zellkulturen GmbH (DSMZ) on 31 Jul. 2019 by SOFAR S.p.A.; with deposit number DSM 33232,
a bacterial strain belonging to the species Bifidobacterium animalis identified as Bifidobacterium animalis subsp. lactis BIIBS01 and deposited at Deutsche Sammlung von Mikroorganismen und Zellkulturen GmbH (DSMZ) on 31 Jul. 2019 by SOFAR S.p.A.; with deposit number DSM 33233,
a bacterial strain belonging to the species Lactobacillus plantarum identified as Lactobacillus plantarum LpIBS01 and deposited at Deutsche Sammlung von Mikroorganismen und Zellkulturen GmbH (DSMZ) on 31 Jul. 2019 by SOFAR S.p.A.; with deposit number DSM 33234, and
mixtures thereof, wherein said composition optionally further comprises at least one food or pharmaceutical grade additive and/or excipient.

2. The composition according to claim 1, wherein said mixture M comprises bacterial strains:
Bifidobacterium breve BbIBS01 DSM 33231,
Bifidobacterium breve BbIBS02 DSM 33232,
Bifidobacterium animalis subsp. lactis BIIBS01 DSM 33233, and
Lactobacillus plantarum LpIBS01 DSM 33234.

3. The composition according to claim 1, wherein said mixture M further comprises at least one further bacterial strain selected from group B comprising:
a bacterial strain identified as Lactobacillus casei DG® deposited at the National Collection of Cultures of Microorganisms Pasteur Institute in Paris under accession number CNCM I-1572 on 5 May 1995 by SOFAR S.p.A.,
a bacterial strain identified as Lactobacillus paracasei LPC-S01™ deposited at Deutsche Sammlung von Mikroorganismen and Zellkulturen GmbH (DSMZ) under accession number DSM 26760 on 15 May 2017 by SOFAR S.p.A. and
a mixture thereof.

4. The composition according to claim 3, wherein said mixture M comprises bacterial strains:
Bifidobacterium breve BbIBS01 DSM 33231,
Bifidobacterium breve BbIBS02 DSM 33232,
Bifidobacterium animalis subsp. lactis BIIBS01 DSM 33233,
Lactobacillus plantarum LpIBS01 DSM 33234, and
Lactobacillus casei DG® CNCM I-1572.

5. The composition according to claim 3, wherein said mixture M comprises bacterial strains:
Bifidobacterium breve BbIBS01 DSM 33231,
Bifidobacterium breve BbIBS02 DSM 33232,
Bifidobacterium animalis subsp. lactis BIIBS01 DSM 33233,
Lactobacillus plantarum LpIBS01 DSM 33234, and
Lactobacillus paracasei LPC-S01™ DSM 26760.

6. The composition according to claim 1, wherein said composition is formulated for oral, sublingual or buccal, transmucosal, topical, rectal, topical cutaneous or topical vaginal use.

7. A method comprising
administering to a subject the composition according to claim 1 as a medicament.

8. A method, comprising
administering to a subject the composition according to claim 1, in an effective amount for preventive and/or curative treatment of functional gastrointestinal diseases, disorders and/or symptoms of the subject.

9. The method according to claim 8, wherein said functional gastrointestinal diseases, disorders and/or symptoms are selected from the group consisting of: irritable bowel syndrome (IBS), dyspepsia, pyrosis, esophageal disorders, stomach disorders and duodenum disorders, small intestinal bacterial overgrowth (SIBO), gastrointestinal disorders with sub-inflammatory conditions; preferably the irritable bowel syndrome (IBS) constipated bowel or irritable bowel syndrome (IBS) diarrhoeic bowel or irritable bowel syndrome (IBS) with alternating constipation and diarrhoea.

10. A method comprising
administering to a subject the composition according to claim 1 an effective amount for preventive and/or curative treatment of inflammatory gastrointestinal diseases, disorders and/or symptoms.

11. The method according to claim 10, wherein said inflammatory gastrointestinal disorders are selected from the group consisting of: chronic inflammatory bowel diseases (IBD), Crohn's disease, ulcerative colitis, microscopic colitis, diverticular disease, diverticulitis, Helicobacter pylori, peptic or gastric ulcer, and duodenal ulcers; preferably selected from chronic inflammatory bowel diseases (IBD), Crohn's disease, ulcerative colitis, microscopic colitis, diverticular disease, and diverticulitis.

12. A bacterial strain belonging to the species Bifidobacterium breve species identified as Bifidobacterium breve BbIBS01, wherein said bacterial strain was deposited, according to the Budapest Treaty, at Deutsche Sammlung von Mikroorganismen und Zellkulturen GmbH (DSMZ) under deposit number DSM 33231 on 31 Jul. 2019 by SOFAR S.p.A.

13. A bacterial strain belonging to the species Bifidobacterium breve identified as Bifidobacterium breve BbIBS02, wherein said bacterial strain was deposited at Deutsche Sammlung von Mikroorganismen und Zellkulturen GmbH (DSMZ) under deposit number DSM 33232 on 31 Jul. 2019 by Sofar S.p.A.

14. A bacterial strain belonging to the species Bifidobacterium animalis identified as Bifidobacterium animalis subsp. lactis BIIBS01, wherein said bacterial strain was deposited at Deutsche Sammlung von Mikroorganismen und Zellkulturen GmbH (DSMZ) under deposit number DSM 33233 on 31 Jul. 2019 by SOFAR S.p.A.

15. A bacterial strain belonging to the species Lactobacillus plantarum identified as Lactobacillus plantarum LpIBS01, wherein said bacterial strain was deposited at Deutsche Sammlung von Mikroorganismen und Zellkulturen GmbH (DSMZ) under deposit number DSM 33234 on 31 Jul. 2019 by SOFAR S.p.A.

16. The composition according to claim 1, wherein said composition is formulated for oral use.

17. The method according to claim 8, wherein said mixture M further comprises at least one further bacterial strain selected from group B comprising
a bacterial strain identified as Lactobacillus casei DG® deposited at the National Collection of Cultures of Microorganisms Pasteur Institute in Paris under accession number CNCM I-1572 on 5 May 1995 by SOFAR S.p.A.,
a bacterial strain identified as Lactobacillus paracasei LPC-S01™ deposited at Deutsche Sammlung von Mikroorganismen und Zellkulturen GmbH (DSMZ) under accession number DSM 26760 on 15 May 2017 by SOFAR S.p.A. and
a mixture thereof.

18. The method of claim 8, wherein the mixture M of the composition consists of bacterial strains:
Bifidobacterium breve BbIBS01 DSM 33231,
Bifidobacterium breve BbIBS02 DSM 33232,
Bifidobacterium animalis subsp. lactis BIIBS01 DSM 33233, and
Lactobacillus plantarum LpIBS01 DSM 33234 and/or Lactobacillus casei DG® CNCM I-1572.

19. The method according to claim 10, wherein said mixture M further comprises at least one further bacterial strain selected from group B comprising
a bacterial strain identified as Lactobacillus casei DG® deposited at the National Collection of Cultures of Microorganisms Pasteur Institute in Paris under accession number CNCM I-1572 on 5 May 1995 by SOFAR S.p.A.,
a bacterial strain identified as Lactobacillus paracasei LPC-S01™ deposited at Deutsche Sammlung von Mikroorganismen and Zellkulturen GmbH (DSMZ) under accession number DSM 26760 on 15 May 2017 by SOFAR S.p.A. and a mixture thereof.

20. The method of claim 10, wherein the mixture M of the composition consists of bacterial strains:
Bifidobacterium breve BbIBS01 DSM 33231,
Bifidobacterium breve BbIBS02 DSM 33232,
Bifidobacterium animalis subsp. lactis BIIBS01 DSM 33233, and
Lactobacillus plantarum LpIBS01 DSM 33234 and/or Lactobacillus casei DG® CNCM I-1572.
